(19) United States
(12) Patent Application Publication
(10) Pub. No.: US 2021/0241044 A1
(43) Pub. Date: Aug. 5, 2021

(54) A KITCHEN ASSISTANT SYSTEM

(71) Applicant: SIMTEK SIMULASYON VE BILISIM TEKN. EGT. MUH. DANIS. TIC. LTD. STI., Ankara (TR)

(72) Inventor: Gul Gul KULALI, Ankara (TR)

(73) Assignee: SIMTEK SIMULASYON VE BILISIM TEKN. EGT. MUH. DANIS. TIC. LTD. STI., Ankara (TR)

(21) Appl. No.: 17/050,485

(22) PCT Filed: Apr. 25, 2019
(86) PCT No.: PCT/TR2019/050276

§ 371 Date: Oct. 26, 2020

(30) Foreign Application Priority Data
Apr. 25, 2018 (TR) 2018/05832

(51) Int. Cl. G06K 9/62; A47J 36/32; G06N 20/00; G06F 16/9035; G06F 16/908; G06F 16/904

(52) U.S. Cl. CPC G06K 9/6263; A47J 36/321; G06N 20/00; G06F 16/904; G06F 16/9035; G06F 16/908; G06K 9/6256

(57) ABSTRACT

The present invention relates to a system which assists a user (K) in the matters of cooking process and kitchen shopping by means of a user device, which is in communication with various kitchenware and gives feedback by observing the cooking progress of the user. The inventive system comprises user device which is used by the user (K), kitchenware, kitchenware Internet of Things (IOT) modules, augmented reality module, camera, image processing module, localization module, suggestion unit, performance evaluation unit, menu database, personal target database and personal preferences database.

### TECHNICAL FIELD

The present invention relates to a system which assists a user in the matters of cooking process and kitchen shopping by means of a user device, which is in communication with various kitchenware and gives feedback by observing the cooking progress of the user.

### BACKGROUND OF THE INVENTION

Today, food preparation and cooking time causes many people to pass over meals and not to be able to eat healthily. People have to note down or keep the missing kitchen materials in mind before shopping. People who permanently cook have difficulty in finding new meals to cook after a while and start cooking the same meals over and over. It is not always possible for people who follow a certain diet to determine and make food. In addition, although people have been cooking for a long time, they cannot follow their cooking progress and cannot get feedback from people eating their food most of the time. However, both saving of time is ensured and it is enabled to make cooking easier, upon smart technologies have come into use in kitchen works as is the case with many areas.

Smart technologies being used in the kitchen are usually focused on kitchen utensils, they don't provide a solution about the cooking process of a person and they only contribute to save time. The fact that the person cannot follow his/her cooking progress causes an obstacle for the said smart technologies to be used completely efficiently. A system which makes comment and evaluation within the scope of smart technologies is not present in the state of the art. There is need for an optimized system which makes choice of menu suitable for the materials available in the kitchen and the time specified, enables to follow multiple diets automatically and to use kitchen materials efficiently, and evaluates the progress of a person in cooking process by observing it.

S The International patent document no. WO0165460, an application in the state of the art, discloses a diet management system according to users' eating habits and targets determined by themselves. The invention can suggest meal on the basis of taste preference data of persons. Distances of food ingredients to users are also taken into consideration. Calorific values of different foods are kept in the inventive system and different food suggestions are made according to physical properties of different users. Users can enter their information such as gender, height, weight, age and target weight, target sizes by creating a profile. Menus compatible with the information entered by the user are chosen while presenting food suggestion for users. The inventive system also creates family groups for users and offers options of exercise programs, restaurant orders and shopping lists as well.

The United States patent document no. US2002029149, another application in the state of the art, discloses a system whereby recipes and food materials are provided to a user over a communication network such that they can be used at certain times, in order to reduce leftover food materials and suggest new food menus promptly. In the inventive system, materials needed for a suggested meal are determined and a shopping list is made. This shopping list can be transmitted to a user device by means of e-mail. In addition, the shopping list can be notified to a market and it is ensured that the materials included in the list can be sent to the user. If a part of the food materials is left, food menus are created based on the leftover food materials for the user. Food menus suited for the conditions designated by the user can be suggested as well. The food menu suggested for the user may consist of picture, video or audio data.

### SUMMARY OF THE INVENTION

One of the objectives of the present invention is to realize a kitchen assistant system which enables to present a kitchen shopping list to a user, to describe the location of materials in the kitchen, to give information about the materials needed for the food to be made.

Another objective of the present invention is to realize a kitchen assistant system which enables to offer menu suggestions in accordance with the user's eating habits determined according to his/her criteria, to receive his/her information about his/her dietary targets by means of various warning notifications.

Another objective of the present invention is to realize a system which enables to observe the cooking progress of the user and to give feedback to the user in accordance with the assessments obtained from the people eating the food.

Another objective of the present invention is to guide a person for the purpose of helping during cooking and to provide professional support.

### DETAILED DESCRIPTION OF THE INVENTION

“A Kitchen Assistant System” realized to fulfil the objective of the present invention is shown in the FIGURE attached, in which:

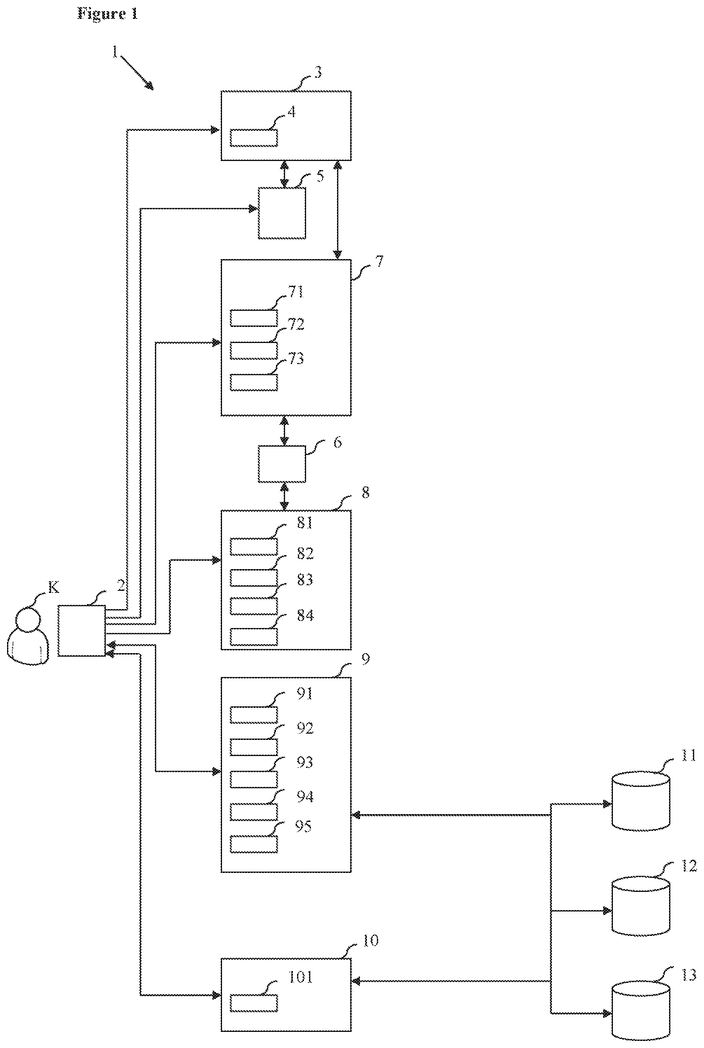

FIG. 1 is a schematic view of the inventive system.

The components illustrated in the FIGURE are individually numbered, where the numbers refer to the following:
1. System
2. User device
3. Kitchenware
4. Kitchenware Internet of Things (IOT) modules
5. Augmented reality module
6. Camera
7. Image processing module
   71. Material control module
   72. Cutting control module
   73. Food preparation control module
8. Localization module
   81. Localization algorithm
   82. Inertial measurement unit-IMU
   83. Camera
   84. Semantic environment map
9. Suggestion unit
   91. Algorithm for menu suggestion
   92. Algorithm for shopping list suggestion
   93. Algorithm for food preparation suggestion
   94. Algorithm for cooking suggestion
   95. Algorithm for food presentation suggestion
10. Performance evaluation unit
   101. Performance evaluation algorithm
11. Menu database
12. Personal target database
13. Personal preferences database
K. User

The inventive kitchen assistant system (1) comprises:
at least one user device (2) which enables the user (K) to communicate with the modules;
kitchenware (3);
kitchenware Internet of Things-OT modules (4) which are located on the kitchenware (3) requested to be interacted with, and which are configured specifically for the kitchenware;
at least one augmented reality module (5) which is configured such that it will provide visual and auditory guidance to the user (K);
at least one camera (6);
at least one image processing module (7) which includes material control module (71), cutting control module (72) and food preparation control module (73);
at least one localization module (8) which is configured such that it will keep the location information of the kitchenware (3) and includes semantic environment map (84), localization algorithm (81), IMU (82) and camera (83);
at least one suggestion unit (9) which includes algorithm for menu suggestion (91), algorithm for shopping list suggestion (92), algorithm for food preparation suggestion (93), algorithm for cooking suggestion (94), algorithm for food presentation suggestion (95);
at least one performance evaluation unit (10) which includes performance evaluation algorithm (101);
at least one menu database (11) whereby menus can be entered by the user (K) or they can be obtained from internet automatically;
at least one personal target database (12) whereby the user (K) can enter his/her preferences and targets and wherein these are kept;
at least one personal preferences database (13) wherein the data that can be updated by the user (K) and the performance evaluation unit (10) are kept.

The user device (2) included in the inventive system (1) is essentially a device such as electronic devices that may be included within the scope of smartphone, tablet, smart glasses, smart home systems having at least internet connection and camera. The user device (2) enables the user (K) interaction with the modules included in the system (1).

The kitchenware Internet of Things-IOT modules (4) are communication units which are located on the kitchenware (3) requested to be interacted with, configured specifically for each kitchenware and run on a communication network.

The augmented reality module (5) included in the inventive system (1) is a unit which is configured such that it will provide visual and auditory guidance to the user (K) and which enables to guide the user (K) by means of contents created in the user device (2) during the process in the kitchen. If the user device (2) is a tablet or smartphone, the user (K) is asked to take his/her mobile device to a suitable location. If the user device (2) is a smart glasses, no positioning is required. It is enough that the glass is worn by the user (K).

The camera (6) included in the inventive system (1) is a unit which is in communication with the other units, provides image so as to be processed in the image processing module (7) and can record during operation of the device. These records can be shared or saved as online and offline over a web service by a user.

The image processing module (7) included in the inventive system (1) is a unit which is in communication with the camera (6) and comprises material control module (71) that controls suitable for the kitchenware (3) selected by the user (K), cutting control module (72) that determines in which size and type of cutting the material is uses a deep learning algorithm, food preparation control module (73) that controls the actions made by the person for the meal and uses a deep learning algorithm. It is detected by the cutting control module (72) which type of cutting—namely, brunoise, jardinière, macédoine, paysanne or dice, matignon, batonnet, julienne, vichy, mirpoix and bouquet garni—is used and whether it is done right or not. Deep learning has one or more layered neural networks and it is an artificial intelligence medium wherein each layer is trained for specific duties and thus output is achieved by processing and gathering complex inputs.

The localization module (8) included in the inventive system (1) is a unit which keeps the semantic environment map (84) wherein the location information of the items and the materials located in the kitchen are comprised and which guides the user (K) by detecting the location of the user (K) by means of the localization algorithm (81) running with the inertial measurement unit-IMU and the camera (83) information. The IMU (82) is a device which enables to measure acceleration and rotation values of moving objects whereon it is placed. In one embodiment of the invention, the localization algorithm (81) included in the localization module (8) uses methods such as Simultaneous Localization and Mapping in order to determine the structure of the kitchen and determine the locations of the kitchenware (3).

The suggestion unit (9) included in the inventive system (1) is a unit which comprises algorithm for menu suggestion (91), algorithm for shopping list suggestion (92), algorithm for food preparation suggestion (93), algorithm for cooking suggestion (94), algorithm for food presentation suggestion (95) consists of artificial intelligence-based modules. The suggestion unit (9) also considers menu selections of the person who will eat. For example, the suggestion unit (9) creates a menu based on the materials available in the kitchen and presents the suggestion to the user (K) over the user device (2). The algorithm for shopping list suggestion (92) creates a list and transmits the list to the user (K) in the event that the materials needed for cooking the meal included in the selected menu are not available in the kitchen. The algorithm for cooking suggestion (94) suggests cooking methods according to the user (K) preferences and/or the menu selected. For example, it offers options such as oven or grill for fish, steaming for vegetables. The decisions of the user (K) about the cooking is transmitted to the performance evaluation unit (10) by the suggestion unit (9). The algorithm for food presentation suggestion (95) creates suggestions about the plate to be used for food presentation, how to put the food on the plate (dish up), suggestions of beverage that can be presented with food and/or table settings. The suggestion unit (9) updates itself in time according to the user (K) selections.

The performance evaluation unit (10) included in the inventive system (1) comprises at least one artificial intelligence module for performance evaluation and has a performance evaluation algorithm (101) and it determines the settings about the kitchenware (3) selected by the user (K) or the system (1) automatically during cooking and so transmits these settings to the kitchenware (3) by means of the kitchenware Internet of Things-IOT modules (4). It creates a time-dependent work list for the menu, preparation and cooking options selected by the user (K) by means of the suggestion unit (9). The performance evaluation algorithm (101) gets feedback from the user (K) over the user device (2) about whether the user (K) has finished the works or not, the problems experienced during the process and the materials. In addition to the information gathered from the user (K), the performance evaluation unit (10) provides notification to the user (K) during cooking by considering the time information and the recipes included in the menu upon receiving data from the image processing module (7) as well. Issues such as whether the meal is cooked within the specified time or not, taste of meal, whether the user (K) used correct materials during cooking or not, comments about cutting types —which are intended for enhancing the performance of the user (K)— are also notified to the user (K). In addition, data are gathered from the persons eating the food after the meal and a suggestion can be made for the user (K) by evaluating these data as up-to-date.

In one embodiment of the invention, evaluation is carried out by the persons participating the meal in the performance evaluation unit (10). Multiple criteria determined by these persons contribute to the result of the evaluation. For this evaluation method, methods are used such as fuzzy-based multi-criteria decision-making which is a logic that is used in cases where the accuracy criterion is not defined explicitly and which can use information comprising graded accuracy.

In another embodiment of the invention, evaluation is carried out in the performance evaluation unit (10) by using the data of recipes specific for the requested cuisines and a deep learning algorithm. Besides, criteria such as performance of the user (K) during cooking, difficulties experienced, whether s/he cooked the meal within the specified time or not, used correct materials or not are also evaluated by the performance evaluation unit (10).

The menu database (11) included in the inventive system (1) is a unit which stores the menus determined or made by the user (K) previously. The user (K) can make changes on these menus and also take new menus from internet. The data can be stored on a cloud structure and shared with other users depending on the user (K) preference. The user (K) can store and share the records that s/he receives during cooking at will.

The personal target database (12) included in the inventive system (1) keeps the weight targets, the diet information and the health information of the user (K). In the event that the healthcare system allows, data can be received over the healthcare system about the user (K). Information of the user (K) such as weight, fat, muscle, blood pressure, cholesterol can be directly transferred from the measurement to the personal target database (12) as up-to-date. Depending on user (K) preference, the data can be shared on a cloud structure and with the persons to be determined by the user (K).

The personal preferences database (13) included in the inventive system (1) stores the personal data that can be updated by the user (K) and the performance evaluation unit (10). Depending on user (K) preference, the data can be shared on a cloud structure and with the persons to be determined by the user (K).

1. A kitchen assistant system (1) comprising
at least one user device (2) which enables the user (K) to communicate with the modules;
kitchenware (3);
kitchenware Internet of Things-IOT modules (4) which are located on the kitchenware (3) requested to be interacted with, and which are configured specifically for the kitchenware;
at least one augmented reality module (5) which is configured such that it will provide visual and auditory guidance to the user (K);
at least one camera (6);
at least one image processing module (7) which includes material control module (71), cutting control module (72) and food preparation control module (73);
at least one localization module (8) which is configured such that it will keep the location information of the kitchenware (3) and includes semantic environment map (84), localization algorithm (81), IMU (82) and camera (83);
at least one suggestion unit (9) which includes algorithm for menu suggestion (91), algorithm for shopping list suggestion (92),
algorithm for food preparation suggestion (93), algorithm for cooking suggestion (94), algorithm for food presentation suggestion (95);
at least one performance evaluation unit (10) which includes performance evaluation algorithm (101);
at least one menu database (11) whereby menus can be entered by the user (K) or they can be obtained from internet automatically;
at least one personal target database (12) whereby the user (K) can enter his/her preferences and targets and wherein these are kept;
at least one personal preferences database (13) wherein the data that can be updated by the user (K) and the performance evaluation unit (10) are kept;
and characterized by
at least one artificial intelligence-based suggestion unit (9) which is configured such that it will suggest menu options and recipes to the user (K) by means of the algorithm for menu suggestion (91) that it has, create a shopping list according to missing kitchen materials by means of the algorithm for shopping list suggestion (92), create preparation steps for cooking the selected meal by means of the algorithm for food preparation suggestion (93), offer suggestions of cooking types by means of the algorithm for cooking suggestion (94), and offer suggestion over the user device (2) about the presentation of the food by means of the algorithm for food presentation suggestion (95), give feedback to the user (K) over the user device (2) during food preparation, cooking and presentation by means of the performance evaluation algorithm (101) that s/he has, follow the cooking progress of the user (K) in a long term by receiving evaluation data from the persons eating food.

2. A system (1) according to claim 1; characterized by the suggestion unit (9) which is configured such that it will suggest menu options and recipes to the user (K) by means of the algorithm for menu suggestion (91) that it has, create a shopping list according to missing kitchen materials by means of the algorithm for shopping list suggestion (92), create preparation steps for cooking the selected meal by means of the algorithm for food preparation suggestion (93), offer suggestions of cooking types by means of the algorithm for cooking suggestion (94), and offer suggestion over the user device (2) about the presentation of the food by means of the algorithm for food presentation suggestion (95); and whereby the preferences can be determined by the user (K).

3. A system (1) according to claim 1; characterized by a performance evaluation unit (10) which is configured such that it will determine the settings about the kitchenware (3) selected by the user (K) or automatically and transmit these settings to the kitchenware (3) by means of the kitchenware Internet of Things-IOT modules (4).

4. A system (1) according to claim 1, characterized by the artificial intelligence-based performance evaluation unit (10) which is configured such that it will create a time-dependent work list for the menu, preparation and cooking options selected by the user (K) by means of the suggestion unit (9); get feedback from the user (K) over the user device (2) about whether the user (K) has finished the works in the work list or not, the problems experienced during the work and the materials by means of the performance evaluation algorithm (101) that it has; and notify these data to the user (K) during cooking or later.

5. A system (1) according to claim 1, characterized by a performance evaluation unit (10) which is configured such that it will provide notification to the user (K) during cooking by considering the time information and the recipes included in the menu upon receiving data from the image processing module (7) with which it is in communication.

6. A system (1) according to claim 1,
characterized by a performance evaluation unit (10) which will gather data from the persons eating food and give feedback to the user (K) by evaluating these data as up-to-date.

7. A system (1) according to claim 1, characterized by a performance evaluation unit (10) which is configured to such that it will evaluate the evaluation data—having multiple criteria received from the persons eating the meal—by using methods such as fuzzy-based multi-criteria decision-making which is a logic that is used in cases where the accuracy criterion is not defined explicitly and that can use information comprising graded accuracy.

8. A system (1) according to claim 1, characterized by a performance evaluation unit (10) which is configured to such that it will carry out evaluation by using the data of recipes specific for the cuisines determined by the user (K) and a deep learning algorithm.
